(19) United States
(12) Patent Application Publication
(10) Pub. No.: US 2022/0096524 A1
(43) Pub. Date: Mar. 31, 2022

(54) METHODS OF PROMOTING HEALTHY CATCH-UP GROWTH

(71) Applicant: ABBOTT LABORATORIES, Abbott Park, IL (US)

(72) Inventors: JOSÉ MARÍA LÓPEZ PEDROSA, Granada (ES); RICARDO RUEDA CABRERA, Granada (ES); MANUEL MANZANO MARTÍN, Granada (ES); JORGE GARCÍA MARTINEZ, Granada (ES); MARÍA CAMPRUBÍ ROBLES, Granada (ES); PILAR BUENO VARGAS, Granada (ES); JOSEPH WALTON, Westerville, OH (US); VIKKIE MUSTAD, Galena, OH (US)

(21) Appl. No.: 17/298,194

(22) PCT Filed: Nov. 27, 2019
(86) PCT No.: PCT/US2019/063669

§ 371 Date: May 28, 2021

(30) Foreign Application Priority Data
Nov. 30, 2018 (EP) 18382874.8

(51) Int. Cl. A61K 31/718; A61K 31/702; A61K 31/7016; A61K 31/7004; A61K 31/733; A61P 3/02; A23L 33/125; A23L 33/00

(52) U.S. Cl. CPC A61K 31/718; A61K 31/702; A61K 31/7016; A61K 31/7004; A23V 2002/00; A61P 3/02; A23L 33/125; A23L 33/40; A61K 31/733

(57) ABSTRACT

A method of promoting healthy catch-up growth in a moderately malnourished individual is provided. The method includes administering to the moderately malnourished individual a nutritional composition that contains a carbohydrate blend, wherein the carbohydrate blend comprises a source of at least one carbohydrate that provides rapidly available glucose, a source of at least one carbohydrate that provides slowly available glucose, and a source of at least one non-digestible carbohydrate or resistant starch.

### CROSS REFERENCE TO RELATED APPLICATIONS

This application claims priority to and the benefit of European Patent Application No. 18382874.8, filed on Nov. 30, 2018, the entire content of which is incorporated herein by reference.

### FIELD

The present disclosure relates to methods of promoting catch-up growth in individuals. More particularly, the present disclosure relates to a method of promoting healthy catch-up growth in a moderately malnourished individual by administering to the moderately malnourished individual a nutritional composition comprising a carbohydrate blend.

### BACKGROUND

The global nutrition community has long been focused on solving the problem of underweight infants and children. However, weight gain itself does not necessarily equate to healthy growth. Indeed, weight gain is unhealthy when the weight gain is rapid and is not accompanied by adequate linear growth. Individuals whose growth faltered during infancy and childhood, but who subsequently showed catch-up growth, are more susceptible to the development of abdominal obesity, type 2 diabetes, and cardiovascular diseases later in life. Catch-up growth is characterized by a disproportionately higher rate in the recovery of body fat than lean tissue, a phenomenon of accelerated fat recovery referred to as “catch-up fat.” The phenomenon of catch-up fat during catch-up growth may lead to hyperinsulinemia and risk of metabolic syndrome later in life.

Accordingly, there is an unmet need for a method of promoting catch-up growth that avoids or reduces the phenomenon of catch-up fat.

### SUMMARY

Disclosed herein are methods of promoting healthy catch-up growth in a moderately malnourished individual by administering to the moderately malnourished individual a nutritional composition comprising a carbohydrate blend.

In accordance with the present disclosure, a method of promoting healthy catch-up growth in a moderately malnourished individual is provided. The method includes administering to the moderately malnourished individual a nutritional composition comprising a carbohydrate blend of: (i) a source of at least one carbohydrate that provides rapidly available glucose; (ii) a source of at least one carbohydrate that provides slowly available glucose; and (iii) a source of at least one non-digestible carbohydrate or resistant starch.

The present disclosure also provides a nutritional composition for use in treating malnourishment in a moderately malnourished individual, the nutritional composition comprising a carbohydrate blend of: (i) a source of at least one carbohydrate that provides rapidly available glucose; (ii) a source of at least one carbohydrate that provides slowly available glucose; and (iii) a source of at least one non-digestible carbohydrate or resistant starch. The malnourishment is treated by promoting healthy catch-up growth.

The present disclosure also provides use of a carbohydrate blend for the manufacture of a medicament for treating malnourishment in a moderately malnourished individual, the carbohydrate blend comprising: (i) a source of at least one carbohydrate that provides rapidly available glucose; (ii) a source of at least one carbohydrate that provides slowly available glucose; and (iii) a source of at least one non-digestible carbohydrate or resistant starch. The malnourishment is treated by promoting healthy catch-up growth.

In accordance with the present disclosure, a method of promoting healthy catch-up growth in a moderately malnourished individual is provided. The method includes administering to the moderately malnourished individual a nutritional composition comprising a protein, a fat, and a carbohydrate blend comprising: (i) a source of at least one carbohydrate that provides rapidly available glucose; (ii) a source of at least one carbohydrate that provides slowly available glucose; and (iii) a source of at least one non-digestible carbohydrate or resistant starch.

The present disclosure also provides a nutritional composition for use in treating malnourishment in a moderately malnourished individual wherein the nutritional composition comprises a protein, a fat, and a carbohydrate blend comprising: (i) a source of at least one carbohydrate that provides rapidly available glucose; (ii) a source of at least one carbohydrate that provides slowly available glucose; and (iii) a source of at least one non-digestible carbohydrate or resistant starch. The malnourishment is treated by promoting healthy catch-up growth.

The present disclosure also provides the use of a nutritional composition for the manufacture of a medicament for treating malnourishment in a moderately malnourished individual, the nutritional composition comprising a protein, a fat, and a carbohydrate blend comprising: (i) a source of at least one carbohydrate that provides rapidly available glucose; (ii) a source of at least one carbohydrate that provides slowly available glucose; and (iii) a source of at least one non-digestible carbohydrate or resistant starch. The malnourishment is treated by promoting healthy catch-up growth.

### BRIEF DESCRIPTION OF THE DRAWINGS

FIG. 1 is a graph depicting body weight measurements of a non-restricted group (NR), a restricted group fed with a diet that predominantly contained sources of carbohydrate that provided rapidly available glucose (RDC), and a restricted group fed with a diet that contained a carbohydrate blend that included a source of carbohydrate that provided rapidly available glucose, a source of carbohydrate that provided slowly available glucose, and a source of non-digestible carbohydrate or resistant starch (SDC) in the study of Example 1;

FIG. 2 is a bar graph depicting fat body mass measured for the RDC group and the SDC group at the end of the study of Example 1;

FIG. 3 is a bar graph depicting resting respiratory quotient (RQ) measured by indirect calorimetry for the RDC group and the SDC group in the study of Example 1;

FIG. 4 is a bar graph depicting the amount of fatty acid synthase (FAS) in white adipose tissue (WAT) for the RDC group and the SDC group in the study of Example 1;

FIG. 5A is a bar graph depicting the change in insulin divided by the change in glucose from a fasting state to a 1-hour post-fed state for the RDC group and the SDC group in the study of Example 1;

FIG. 5B is a bar graph depicting the expression of glucose transporter 4 (GLUT4) in muscle for the RDC group and the SDC group in the study of Example 1;

FIG. 6A is a bar graph depicting the amount of lean body mass measured for the RDC group and the SDC group in the study of Example 1;

FIG. 6B is a bar graph depicting grip strength measurements for the RDC group and the SDC group in the study of Example 1;

FIG. 7 is a bar graph depicting the expression of ATP synthase (ATP5B) in muscle for the RDC group and the DSC group in the study of Example 1;

FIG. 8 is a graph depicting cumulative energy (kcal) utilization in an RDC diet group and an SDC diet group in the study of Example 2;

FIG. 9A is a graph depicting exogenous carbohydrate energy (kcal) utilization in an RDC diet group and an SDC diet group in the study of Example 2;

FIG. 9B is a graph depicting endogenous carbohydrate energy (kcal) utilization in an RDC diet group and an SDC diet group in the study of Example 2;

FIG. 9C is a graph depicting endogenous fat energy (kcal) utilization in an RDC diet group and an SDC diet group in the study of Example 2;

FIG. 9D is a bar graph depicting the relative area under the curve (AUC) for energy utilization across the rest phase for the RDC diet group and the SDC diet group (labelled as CHO-2) in the study of Example 2;

FIG. 9E is a bar graph depicting the relative area under the curve (AUC) for energy utilization across the rest+exercise phases for the RDC diet group and the SDC diet group (labelled as CHO-2) in the study of Example 2;

FIG. 9F is a bar graph depicting the relative area under the curve (AUC) for energy utilization across the rest+exercise+recovery phases for the RDC diet group and the SDC diet group (labelled as CHO-2) in the study of Example 2;

FIG. 10A is a graph depicting the concentration of capillary glucose and insulin for the RDC diet group and the SDC diet group in the study of Example 2; and

FIG. 10B is a graph depicting glucose responses of the RDC diet group and the SDC diet group in the study of Example 2.

### DETAILED DESCRIPTION

Disclosed herein are methods of promoting healthy catch-up growth in a moderately malnourished individual. While the present disclosure describes certain embodiments of the methods in detail, the present disclosure is to be considered exemplary and is not intended to be limited to the disclosed embodiments. Also, certain elements or features of embodiments disclosed herein are not limited to a particular embodiment, but instead apply to all embodiments of the present disclosure.

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

To the extent that the term “includes” or “including” is used in the description or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use.

The methods of the present disclosure can comprise, consist of, or consist essentially of the essential elements of the disclosure as described herein, as well as any additional or optional element described herein or which is otherwise useful in nutritional applications.

All percentages, parts, and ratios as used herein are by weight of the total composition unless otherwise specified. All such weights as they pertain to listed ingredients are based on the active level and, therefore, do not include solvents, by-products, or other components that may be included in commercially available materials, unless otherwise specified.

All ranges and parameters, including but not limited to percentages, parts, and ratios, disclosed herein are understood to encompass any and all sub-ranges assumed and subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, and 10) contained within the range.

Any combination of method or process steps as used herein may be performed in any order, unless otherwise specified or clearly implied to the contrary by the context in which the referenced combination is made.

The term “individual” as used herein, refers generally to a preterm infant, infant, toddler, or child.

The term “infant” as used herein, refers generally to individuals up to 36 months of age, actual or corrected.

The term “preterm infant” as used herein, refers to infants born at less than 37 weeks gestation, infants that have a birth weight of less than 2,500 grams, or both.

The term “nutritional composition” as used herein, unless otherwise specified, refers to synthetic formulas including nutritional liquids, nutritional powders, nutritional solids, nutritional semi-solids, nutritional semi-liquids, nutritional supplements, and any other nutritional food product as known in the art. The nutritional powders may be reconstituted to form a nutritional liquid, all of which comprise one or more of fat, protein, and carbohydrate and are suitable for oral consumption by a human. The term “nutritional composition” does not include human breast milk and does not refer to supplemented milk.

The term “nutritional liquid” as used herein, unless otherwise specified, refers to nutritional compositions in ready-to-drink liquid form, concentrated form, and nutritional liquids made by reconstituting the nutritional powders described herein prior to use.

The term “nutritional powder” as used herein, unless otherwise specified, refers to nutritional compositions in flowable or scoopable form that can be reconstituted with water or another aqueous liquid prior to consumption and includes both spray dried and dry mixed/dry blended powders.

The term “shelf stable” as used herein, unless otherwise specified, refers to a nutritional composition that remains commercially stable after being packaged and then stored at 18-24° C. for at least 3 months, including from about 3 months to about 24 months, and also including from about 3 months to about 18 months.

The term “catch-up growth” as used herein, unless otherwise specified, refers to a remedial level or rate of growth or development to achieve a basal level of growth following a transient period of growth restriction or inhibition.

The term “healthy catch-up growth” as used herein, unless otherwise specified, refers to catch-up growth in which the phenomenon of catch-up fat is avoided or reduced.

The term “catch-up fat” as used herein, unless otherwise specified, refers to a disproportionately higher rate in the recovery of body fat than lean tissue during a period of catch-up growth.

The terms “moderate malnutrition” and “moderately malnourished” as used herein, unless otherwise specified, refer to a weight-for-age between −3 and −2 z-scores below the median of the World Health Organization (WHO) child growth standards. Moderate malnutrition can be due to a low weight-for-height (wasting) or a low height-for-age (stunting) or to a combination of both. Similarly, moderate wasting and stunting are defined as a weight-for-height and height-for-age, respectively, between −3 and −2 z-scores.

The methods of promoting healthy catch-up growth in a moderately malnourished individual according to the present disclosure are based on the discovery that administration of a nutritional composition having a particular blend of carbohydrates improves metabolic flexibility in the moderately malnourished individual such that catch-up fat is avoided or reduced during catch-up growth.

An important factor contributing to catch-up fat is a reduction in fat oxidation and an increase in carbohydrate oxidation. This shift towards a preferential metabolic use of carbohydrates rather than the adipose tissue fat pads with a concomitant lower glucose utilization in the skeletal muscle results in reduced insulin sensitivity and compensatory hyperinsulinemia. This in turn serves to redirect spared glucose toward de novo lipogenesis in adipose tissue, a function that coordinates glucose spared for catch-up fat with glucose homeostasis. The phenomenon of impaired metabolic flexibility for the purpose of sparing glucose utilization away from skeletal muscle toward adipose tissue represents a central event in the link between catch-up fat, hyperinsulinemia, and risk for later metabolic syndrome.

Metabolic flexibility is the capacity to utilize fat and carbohydrate fuels and to transition between them in response to changes in dietary intake or circulating substrate concentrations. A failure in metabolic flexibility may lead not only to the metabolic sequelae of insulin resistance, but to the obese state. Accordingly, controlling metabolic flexibility to utilize fat and carbohydrate fuels in a more balanced way can promote healthy catch-up growth by avoiding or reducing catch-up fat.

The methods of promoting healthy catch-up growth in a moderately malnourished individual according to the present disclosure comprise the administration of a nutritional composition to the moderately malnourished individual. The nutritional composition administered to the moderately malnourished individual comprises a carbohydrate blend. In embodiments of the method, the nutritional composition administered to the moderately malnourished individual comprises one or more of a protein and a fat, in addition to the carbohydrate blend.

The carbohydrate blend comprises a source of at least one carbohydrate that provides rapidly available glucose, a source of at least one carbohydrate that provides slowly available glucose, and a source of at least one non-digestible carbohydrate or resistant starch. Carbohydrates that provide rapidly available glucose are rapidly absorbed in the duodenum and proximal regions of the small intestine leading to a rapid elevation of blood glucose and usually a subsequent episode of hypoglycemia. Carbohydrates that provide slowly available glucose are steadily but completely digested resulting in prolonged glucose release from the lumen of the small intestine into the blood stream. Non-digestible carbohydrates or resistant starches are carbohydrates or factions thereof that are not digested in the upper gastrointestinal tract, but are fermented in the large intestine by the gut microflora, producing short chain fatty acids that provide additional energy to the body.

The terms “rapidly available glucose” and “slowly available glucose” as used herein reflect the rate at which glucose becomes available for absorption in the human small intestine according to the in vitro method developed by Englyst et al. (Am J Clin Nutr (1999), Vol. 69, pp 448-454), which is incorporated by reference herein. This in vitro method characterizes dietary carbohydrates with regard to their chemical composition and likely gastrointestinal fate. The glycemic carbohydrate fraction that is available for absorption in the small intestine is measured as the sum of sugars and starch (including maltodextrins) and excludes resistant starch. The Englyst method determines rapidly available glucose, slowly available glucose, and starch fractions by measuring the amount of glucose released from a carbohydrate or carbohydrate source during timed incubation (20 minutes and 120 minutes) with digestive enzymes under standardized conditions. In accordance with the Englyst method, for a carbohydrate or carbohydrate source the amount of glucose measured at 20 minutes (G₂₀) represents “rapidly available glucose,” whereas the difference between the amount of glucose measured at 120 minutes (G₁₂₀) and the G₂₀ value (i.e., G₁₂₀−G₂₀) represents “slowly available glucose.” The Englyst method also enables the calculation of: (i) rapidly digestible starch, which contributes to the amount of rapidly available glucose; (ii) slowly digestible starch, which contributes to the amount of slowly available glucose; (iii) total starch; and (iv) resistant starch.

In accordance with the present disclosure, the carbohydrate blend comprises a source of at least one carbohydrate that provides rapidly available glucose. In embodiments, the source of at least one carbohydrate that provides rapidly available glucose comprises one or more of: (i) a monosaccharide; (ii) a glucose unit and a fructose unit joined by an α-1, β-2 glycosidic linkage; (iii) a glucose unit and a galactose unit joined by a β (1,4) glycosidic linkage; (iv) glucose units joined by α (1,4) glycosidic linkages; (v) glucose units joined by α (1,6) glycosidic linkages; and (vi) an oligosaccharide having a random mixture of α (1,2), α (1,3), α (1,4), and α (1,6) glycosidic linkages.

In embodiments, the source of at least one carbohydrate that provides rapidly available glucose in the carbohydrate blend comprises a monosaccharide. Exemplary monosaccharides suitable for use in the carbohydrate blend to provide rapidly available glucose include, but are not limited to, glucose, fructose, tagatose, galactose, mannose, and ribose.

In embodiments, the source of at least one carbohydrate that provides rapidly available glucose in the carbohydrate blend comprises a glucose unit and a fructose unit joined by an α-1, β-2 glycosidic linkage. One example of a carbohydrate that includes a glucose unit and a fructose unit joined by an α-1, β-2 glycosidic linkage is sucrose.

In embodiments, the source of at least one carbohydrate that provides rapidly available glucose in the carbohydrate blend comprises a galactose unit and a glucose unit joined by a β (1,4) glycosidic linkage. One example of a carbohydrate that includes a galactose unit and a glucose unit joined by a β (1,4) glycosidic linkage is lactose.

In embodiments, the source of at least one carbohydrate that provides rapidly available glucose in the carbohydrate blend comprises glucose units joined by α (1,4) glycosidic linkages. Exemplary carbohydrates or carbohydrate sources having glucose units joined by α (1,4) glycosidic linkages include, but are not limited to, maltose, maltodextrin, and starch.

In embodiments, the source of at least one carbohydrate that provides rapidly available glucose in the carbohydrate blend comprises glucose units joined by α (1,6) glycosidic linkages. One example of a carbohydrate having glucose units joined by α (1,6) glycosidic linkages is isomaltose.

In embodiments, the source of at least one carbohydrate that provides rapidly available glucose in the carbohydrate blend comprises oligosaccharides having a random mixture of α (1,2), α (1,3), α (1,4), and α (1,6) glycosidic linkages. One example of a source of carbohydrate that includes oligosaccharides having a random mixture of α (1,2), α (1,3), α (1,4), and α (1,6) glycosidic linkages is isomalto-oligosaccharides. Suitable isomalto-oligosaccharides that provide rapidly available glucose include mixtures of oligosaccharides with a degree of polymerization (DP) of 3 or greater including, but not limited to, isomaltose, panose, maltotetraose isomaltotriose, isomaltotetraose, maltopentaose, isomaltopentaose, maltohexaose, isomaltohexaose, maltoheptaose, isomaltoheptaose, maltooctaose, isomaltooctaose, maltononaose, and isomaltononaose.

In accordance with the present disclosure, the carbohydrate blend comprises a source of at least one carbohydrate that provides slowly available glucose. In embodiments, the source of at least one carbohydrate that provides slowly available glucose comprises one or more of: (i) a glucose unit and a fructose unit joined by an α (1,6) glycosidic linkage; (ii) two glucose units joined by an α (1,1) glycosidic linkage; (iii) a glucose unit and a fructose unit joined by an α (1,5) glycosidic linkage; and (iv) an oligosaccharide having alternating α (1,3) and α (1,6) glycosidic linkages.

In embodiments, the source of at least one carbohydrate that provides slowly available glucose in the carbohydrate blend comprises a glucose unit and a fructose unit joined by an α (1,6) glycosidic linkage. One example of a carbohydrate having a glucose unit and a fructose unit joined by an α (1,6) glycosidic linkage is isomaltulose.

In embodiments, the source of at least one carbohydrate that provides slowly available glucose in the carbohydrate blend comprises two glucose units joined by an α (1,1) glycosidic linkage. One example of a carbohydrate having two glucose units joined by an α (1,1) glycosidic linkage is trehalose.

In embodiments, the source of at least one carbohydrate that provides slowly available glucose in the carbohydrate blend comprises a glucose unit and a fructose unit joined by an α (1,5) glycosidic linkage. One example of a carbohydrate having a glucose unit and a fructose unit joined by an α (1,5) glycosidic linkage is leucrose. Leucrose is a disaccharide that is present in sucromalt.

In embodiments, the source of at least one carbohydrate that provides slowly available glucose in the carbohydrate blend comprises oligosaccharides having alternating α (1,3) and α (1,6) glycosidic linkages. One example of a source of carbohydrate that includes oligosaccharides having alternating α (1,3) and α (1,6) glycosidic linkages is sucromalt.

In accordance with the present disclosure, the carbohydrate blend comprises a source of at least one non-digestible carbohydrate or resistant starch. In embodiments, the source of at least one non-digestible carbohydrate or resistant starch comprises one or more of: (i) oligosaccharides having a random mixture of α (1,2), α (1,3), α (1,4), and β glycosidic linkages; (ii) saccharides having linear chains of 2 to 60 fructose units joined by α (2,1) glycosidic linkages or fructose polymers joined by β (2,1) glycosidic linkages; and (iii) oligosaccharides having a random mixture of α (1,2), α (1,3), α (1,4), and α (1,6) glycosidic linkages.

In embodiments, the source of at least one non-digestible carbohydrate or resistant starch (including maltodextrin) comprises oligosaccharides having a random mixture of α (1,2), α (1,3), α (1,4), and β glycosidic linkages. One example of a source of carbohydrate that includes oligosaccharides having a random mixture of α (1,2), α (1,3), α (1,4), and β glycosidic linkages is resistant maltodextrin. The mixture of oligosaccharides making up resistant maltodextrin are produced by pyrolysis and enzymatic hydrolysis of starch (e.g., corn, wheat, rice, potato) and have a molecular weight of about 2,000 Daltons. Examples of commercially available resistant maltodextrin include Nutriose® resistant maltodextrin from Roquette America, Inc. (Geneva, Ill.) and Fibersol® digestion resistant maltodextrin from ADM/Matsutani LLC (Itasca, Ill.).

In embodiments, the source of at least one non-digestible carbohydrate or resistant starch comprises saccharides having linear chains of 2 to 60 fructose units or fructose polymers joined by β (2,1) glycosidic linkages. Exemplary carbohydrates and carbohydrate sources that include saccharides having linear chains of 2 to 60 fructose units or fructose polymers joined by β (2,1) glycosidic linkages include, but are not limited to, inulin and fructooligosaccharide.

In embodiments, the source of at least one non-digestible carbohydrate or resistant starch comprises oligosaccharides having a random mixture of α (1,2), α (1,3), α (1,4), and α (1,6) glycosidic linkages. One example of a source of carbohydrate that includes oligosaccharides having a random mixture of α (1,2), α (1,3), α (1,4), and α (1,6) glycosidic linkages is isomalto-oligosaccharides. The isomalto-oligosaccharides may be any one or more of the isomalto-oligosaccharides previously described herein.

In embodiments, the carbohydrate blend comprises: (i) a source of at least one carbohydrate that provides rapidly available glucose selected from one or more of glucose, fructose, galactose, mannose, ribose, sucrose, lactose, maltose, isomaltose, maltodextrin, starch, or isomalto-oligosaccharides; (ii) a source of at least one carbohydrate that provides slowly available glucose selected from one or more of isomaltulose, trehalose, leucrose, or sucromalt; and (iii) a source of at least one non-digestible or resistant starch selected from one or more of resistant starch, fructooligosaccharides, inulin, or isomalto-oligosaccharides. In embodiments, the carbohydrate blend comprises: (i) a source of at least one carbohydrate that provides rapidly available glucose selected from one or more of maltodextrin, isomaltose, or isomalto-oligosaccharides; (ii) a source of at least one carbohydrate that provides slowly available glucose selected from one or more of isomaltulose, sucromalt, trehalose, or leucrose; and (iii) a source of at least one non-digestible or resistant starch selected from one or more of resistant starch, fructooligosaccharides, inulin, or isomalto-oligosaccharides. In embodiments, the carbohydrate blend comprises: (i) a source of at least one carbohydrate that provides rapidly available glucose selected from one or more of maltodextrin or isomalto-oligosaccharides; (ii) a source of at least one carbohydrate that provides slowly available glucose selected from one or more of isomaltulose or sucromalt; and (iii) a source of at least one non-digestible or resistant starch selected from one or more of resistant starch, fructooligosaccharides, inulin, or isomalto-oligosaccharides.

Certain sources of carbohydrates used in the carbohydrate blend may include carbohydrates or fractions thereof that provide more than one category of glucose availability (i.e., rapidly available glucose, slowly available glucose, and non-available glucose (e.g., non-digestible carbohydrate or resistant starch)). Thus, in accordance with the methods of the present disclosure, a source of carbohydrate in the carbohydrate blend can include one or more of a carbohydrate that provides rapidly available glucose, a carbohydrate the provides slowly available glucose, and a non-digestible carbohydrate or resistant starch. For example, a source of carbohydrate that includes a carbohydrate that provides rapidly available glucose and a non-digestible carbohydrate or resistant starch (e.g., through a fiber fraction, which is a non-digestible carbohydrate or resistant starch) is isomalto-oligosaccharides. An example of a source of carbohydrate that includes a carbohydrate that provides rapidly available glucose and a carbohydrate that provides slowly available glucose is sucromalt. Accordingly, a single source of carbohydrate may be used in the methods of the present disclosure to provide one or more than one of a carbohydrate that provides rapidly available glucose, a carbohydrate that provides slowly available glucose, and a non-digestible carbohydrate or resistant starch.

In embodiments, the source of at least one carbohydrate of the carbohydrate blend that provides rapidly available glucose provides from 4% to 71% of the total calories supplied by carbohydrates in the nutritional composition, the source of at least one carbohydrate of the carbohydrate blend that provides slowly available glucose provides from 25% to 86% of the total calories supplied by carbohydrates in the nutritional composition, and the source of at least one non-digestible carbohydrate or resistant starch of the carbohydrate blend provides from 3% to 20% of the total calories supplied by carbohydrates in the nutritional composition. A carbohydrate blend having such a distribution of carbohydrates with the specified glucose availability accelerates the development of healthy catch-up growth by establishing and maintaining a more balanced use of carbohydrates and fats as energy sources.

As previously mentioned, in embodiments of the method of the present disclosure, the nutritional composition administered to the moderately malnourished individual comprises one or more of a protein and a fat, in addition to the carbohydrate blend. In embodiments of the method of the present disclosure, the nutritional composition administered to the moderately malnourished individual comprises a protein, a fat, and a carbohydrate blend as previously described.

As previously mentioned, a nutritional composition for use in treating malnourishment in a moderately malnourished individual, comprises one or more of a protein and a fat, in addition to a carbohydrate blend as previously described.

In addition, in use of a nutritional composition for the manufacture of a medicament for treating malnourishment in a moderately malnourished individual, the nutritional composition comprises one or more of a protein and a fat, in addition to a carbohydrate blend as previously described.

The amount of carbohydrates in the nutritional composition will typically range from about 5% to about 75%, including from about 15% to about 70%, including from about 30% to about 65%, by weight of the nutritional composition, on a dry weight basis. When present, the amount of fat in the nutritional composition will typically range from about 1% to about 30%, including from about 2% to about 15%, and also including from about 3% to about 10%, by weight of the nutritional composition, on a dry weight basis. When present, the amount of protein in the nutritional composition will typically range from about 0.5% to about 30%, including from about 1% to about 25%, and also including from about 10% to about 20%, by weight of the nutritional composition, on a dry weight basis.

The amount of any or all of the carbohydrates, fats, and proteins in any of the nutritional compositions described herein may also be characterized as a percentage of total calories in the nutritional composition as set forth in the following table. The macronutrients for nutritional compositions suitable for use in accordance with the methods of the present disclosure are most typically formulated within any of the caloric ranges (embodiments A-F) described in the following table (each numerical value is preceded by the term “about”).

TABLE 1
Exemplary macronutrient profiles of nutritional compositions

| | Embodiment A | Embodiment B | Embodiment C |
|---|---|---|---|
| Nutrient | (% Total Cal.) | (% Total Cal.) | (% Total Cal.) |
| Carbohydrate | 1-98 | 2-96 | 10-75 |
| Protein | 1-98 | 2-96 | 5-70 |
| Fat | 1-98 | 2-96 | 20-85 |
| | Embodiment D | Embodiment E | Embodiment F |
| | (% Total Cal.) | (% Total Cal.) | (% Total Cal.) |
| Carbohydrate | 30-50 | 25-50 | 25-50 |
| Protein | 15-35 | 10-30 | 5-30 |
| Fat | 35-55 | 1-20 | 2-20 |

The nutritional composition used in accordance with the methods of the present disclosure may comprise a fat or a source of fat. The fat or source of fat used in the nutritional composition may be derived from various sources including, but not limited to, plants, animals, and combinations thereof.

Sources of fat that are suitable for use in the nutritional composition include, but are not limited to, coconut oil, fractionated coconut oil, soy oil, high oleic soy oil, corn oil, olive oil, safflower oil, high oleic safflower oil, medium chain triglyceride oil (MCT oil), high gamma linolenic (GLA) safflower oil, sunflower oil, high oleic sunflower oil, palm oil, palm kernel oil, palm olein, canola oil, high oleic canola oil, marine oils, fish oils (e.g., tuna oil), algal oils, borage oil, cottonseed oil, fungal oils, eicosapentaenoic acid (EPA), docosahexaenoic acid (DHA), arachidonic acid (ARA), conjugated linoleic acid (CLA), alpha-linolenic acid, interesterified oils, transesterified oils, structured lipids, and combinations thereof.

Generally, the fat or source of fat used in the nutritional composition provides fatty acids needed both as an energy source and for the healthy development of the individual. The source of fat typically comprises triglycerides, although the source of fat may also comprise diglycerides, monoglycerides, phospholipids (e.g., lecithin) and free fatty acids. Fatty acids provided by the source of fat in the nutritional composition include, but are not limited to, capric acid, lauric acid, myristic acid, palmitic acid, palmitoleic acid, stearic acid, oleic acid, linoleic acid, alpha-linolenic acid, ARA, EPA, DHA, and combinations thereof. The nutritional composition administered in accordance with the methods of the present disclosure may include any individual source of fat or combination of the various sources of fat listed above.

The nutritional composition used in accordance with the methods of the present disclosure may comprise a protein or a source of protein. The protein or source of protein used in the nutritional composition may be a plant-based protein, an animal-based protein, a milk-based protein, and combinations of the foregoing. The protein may be intact, partially hydrolyzed (i.e., a degree of hydrolysis of less than 20%), or extensively hydrolyzed (i.e., a degree of hydrolysis of at least 20%). The protein may also include at least one free amino acid.

Plant-based proteins suitable for use in the nutritional composition include, but are not limited to, soy protein, pea protein, rice protein, and potato protein. Animal-based proteins suitable for use in the nutritional composition include, but are not limited to, poultry protein (e.g., chicken protein), collagen, fish protein, ovine protein, porcine protein, and bovine protein. Milk-based proteins suitable for use in the nutritional composition include, but are not limited to, whole cow's milk, partially or completely defatted milk, milk protein concentrates, milk protein isolates, nonfat dry milk, condensed skim milk, whey protein concentrates, whey protein isolates, acid caseins, sodium caseinates, calcium caseinates, and potassium caseinates. The source of protein may be certified organic (e.g., USDA organic) or non-organic, and also may be non-genetically modified (non-GMO) or genetically modified. The nutritional composition for use in accordance with the methods of the present disclosure may include any individual source of protein or combination of the various sources of protein listed above.

In addition, the source of protein in the nutritional composition can also include one or more free amino acids. Free amino acids that may be used in the nutritional composition include, but are not limited to, L-lysine, L-tryptophan, L-glutamine, L-tyrosine, L-methionine, L-cysteine, taurine, L-arginine, L-leucine, L-isoleucine, L-valine, and L-carnitine.

The nutritional composition used in accordance with the methods of the present disclosure may comprise vitamins and minerals. In embodiments, the nutritional composition comprises at least one of vitamin A, vitamin D, vitamin E, vitamin K, thiamine, riboflavin, pyridoxine, vitamin B₁₂, niacin, folic acid, pantothenic acid, biotin, vitamin C, choline, and inositol. In embodiments, the nutritional composition comprises at least one of calcium, phosphorus, magnesium, iron, zinc, manganese, copper, sodium, potassium, molybdenum, chromium, selenium, chloride, and iodine.

The nutritional composition used in accordance with the methods of the present disclosure may also include a variety of optional ingredients that may modify the physical, chemical, or processing characteristics of the nutritional composition, or merely serve as additional nutritional component. In embodiments, the nutritional composition may comprise one or more of a probiotic, a nucleotide, a nucleoside, and a carotenoid (e.g., lutein, beta-carotene, lycopene, zeaxanthin).

The nutritional composition for use in accordance with the methods of the present disclosure may have a caloric density tailored to the nutritional needs of the ultimate user. In embodiments of the present disclosure, the nutritional composition has a caloric density of at least 1 kcal/mL. In embodiments of the present disclosure, the nutritional composition has a caloric density of 1 kcal/mL to 2 kcal/mL, including from 1.1 kcal/mL to 2 kcal/mL, from 1.15 kcal/mL to 1.9 kcal/mL, from 1.2 kcal/mL to 1.8 kcal/mL, and also including from 1.25 kcal/mL to 1.5 kcal/mL.

The nutritional composition for use in accordance with the methods of the present disclosure may be formulated and administered in any known or otherwise suitable oral product form. Any solid, liquid, semi-solid, and semi-liquid, or powder product form, including combinations or variations thereof, are suitable for use herein, provided that such forms allow for safe and effective oral delivery to the individual of the essential ingredients disclosed herein.

The nutritional composition may be in any product form comprising the ingredients described herein, and which is safe and effective for oral administration. The nutritional composition may be formulated to include only the ingredients described herein, or may be modified with optional ingredients to form a number of different product forms.

In embodiments, the nutritional composition administered in accordance with the methods of the present disclosure is formulated as a liquid nutritional composition. Liquid nutritional compositions include both concentrated and ready-to-feed nutritional liquids. These nutritional liquids are most typically formulated as suspensions or emulsions, although other liquid forms are within the scope of the present disclosure. Nutritional compositions in the form of emulsions suitable for use may be aqueous emulsions comprising proteins, fats, and carbohydrates. These emulsions are generally flowable or drinkable liquids at from about 1° C. to about 25° C. and are typically in the form of oil-in-water, water-in-oil, or complex aqueous emulsions, although such emulsions are most typically in the form of oil-in-water emulsions having a continuous aqueous phase and a discontinuous oil phase.

The nutritional liquid may be and typically are shelf stable. The nutritional liquid typically contains up to about 95% by weight of water, including from about 50% to about 95%, also including from about 60% to about 90%, and also including from about 70% to about 85%, of water by weight of the nutritional liquid. The nutritional liquid may have a variety of product densities, but most typically have a density greater than about 1.03 g/mL, including greater than about 1.04 g/mL, including greater than about 1.055 g/mL, including from about 1.06 g/mL to about 1.12 g/mL, and also including from about 1.085 g/mL to about 1.10 g/mL. The nutritional liquid may have a pH ranging from about 2.5 to about 8, but are most advantageously in a range of from about 4.5 to about 7.5, including from about 5.5 to about 7.3, including from about 6.2 to about 7.2.

Although the serving size for the nutritional liquid can vary depending upon a number of variables, a typical serving size is generally at least about 1 mL, or even at least about 2 mL, or even at least about 5 mL, or even at least about 10 mL, or even at least about 25 mL, including ranges from about 1 mL to about 360 mL, including from about 30 mL to about 250 mL, and including from about 60 mL to about 240 mL.

In embodiments, the nutritional composition administered in accordance with the methods of the present disclosure is formulated as a nutritional solid. The nutritional solids may be in any solid form but are typically in the form of flowable or substantially flowable particulate compositions, or at least particulate compositions. Particularly suitable nutritional solid product forms include spray dried, agglomerated, and/or dryblended powder compositions. The compositions can easily be scooped and measured with a spoon or similar other device, and can easily be reconstituted with a suitable aqueous liquid, typically water, to form a nutritional composition for immediate oral or enteral use. In this context, “immediate” use generally means within about 48 hours, most typically within about 24 hours, preferably right after reconstitution.

As previously mentioned, the methods of promoting healthy catch-up growth in a moderately malnourished individual according to the present disclosure comprise the administration of a nutritional composition to the moderately malnourished individual. The nutritional composition comprises a carbohydrate blend as previously described. Any of the various nutritional compositions described herein can be administered to the moderately malnourished individual in accordance with the methods of the present disclosure.

As previously mentioned, a nutritional composition for use in treating malnourishment in a moderately malnourished individual, comprises a carbohydrate blend as previously described.

In addition, in use of a nutritional composition for the manufacture of a medicament for treating malnourishment in a moderately malnourished individual, the nutritional composition comprises a carbohydrate blend as previously described.

In embodiments of the method the present disclosure, the moderately malnourished individual may be an infant, a preterm infant, a toddler, or a child. In embodiments of the method of the present disclosure, the moderately malnourished individual suffers from one or more of stunting and wasting. As briefly mentioned above, stunting is characterized by a low height-for-age, such as a height-for-age z-score of less than −2 based on the WHO child growth standards median. Similarly, wasting is characterized by a low weight-for-height, such as a weight-for-height z-score of less than −2 based on the WHO child growth standards median. Stunting and wasting may be caused by a number of factors including, but not limited to, malnutrition, undernutrition, poor gut health, infectious disease and compromised immune function. In the methods of the present disclosure, the moderately malnourished individual is an infant or a preterm infant who suffers from stunting, and the stunting is caused by one or more of malnutrition, undernutrition, poor gut health, infectious disease, and compromised immune function.

In accordance with the methods of the present disclosure, administration of a nutritional composition having a carbohydrate blend as described herein improves metabolic flexibility such that catch-up fat is avoided or reduced during catch-up growth, resulting in growth characterized by the accumulation of less fat mass—in other words, healthy catch-up growth. The methods of promoting healthy catch-up growth of the present disclosure may also reduce the risk that an individual will develop conditions later in life including, but not limited to, obesity, insulin resistance, type 2 diabetes, cardiovascular disease, and metabolic syndrome.

### Examples

The following examples are presented to provide a better understanding of the methods of the present disclosure. The examples are for purposes of illustration only and are not intended to limit the scope of the present disclosure.

Example 1—A study was conducted to evaluate the effect of chronic administration of carbohydrate blends on healthy growth associated outcomes using an animal model of growth retardation or nutritional dwarfing.

The nutritional dwarfing model is based on developing a nutritional stress in weanling male rats placed on restricted intake (30% of normal intake) of the control diet for four weeks. The restriction period was followed by another four weeks with full access to the experimental diets.

During the restriction period, animals were divided into two groups. Non-restricted rats (NR group) were fed with a standard rodent diet (AIN93M) ad libitum during the entire study period. Restricted rats (RR group) received 70% of the amount of food consumed by the NR group. After the four-week food restriction period, the RR group was fed ad libitum with different humanized experimental diets (RDC group and SDC group) for four weeks (the refeeding period). The RDC and SDC diets were designed to be isoenergetic. However, it should be noted that the RDC diet predominantly contains sources of carbohydrate that provide rapidly available glucose while the SDC diet contains a carbohydrate blend that includes a source of carbohydrate that provides rapidly available glucose, a source of carbohydrate that provides slowly available glucose, and a source of non-digestible carbohydrate or resistant starch in accordance with the present invention. The AIN93M diet is a purified rodent diet structured to provide nutrients in concentrations required just to maintain adult rat or mouse populations. The compositions of the diets are provided in Table 2 below.

TABLE 2
Composition of Diets

| | AIN93M | RDC | SDC |
|---|---|---|---|
| Macronutrients | Diet | Diet | Diet |
| Carbohydrates (CHO) (g/100 g diet) | 78.5 | 67 | 67 |
| Sucrose (g/100 g CHO) | 13 | 32 | |
| Isomaltulose (g/100 g CHO) | | | 26.4 |
| Sucromalt (g/100 g CHO) | | | 22.1 |
| Cornstarch (g/100 g CHO) | 61 | | |
| MD₂₉ (g/100 g CHO) | | 64 | 23 |
| MD₉₋₁₆ (g/100 g CHO) | 20 | | |
| IMOs (g/100 g CHO) | | | 11.5 |
| Resistant starch (g/100 g CHO) | | | 10 |
| FOS (g/100 g CHO) | | 4 | |
| Inulin:FOS (g/100 g CHO) | | | 7 |
| Cellulose (g/100 g CHO) | 5 | | |
| Protein (g/100 g diet) | 14 | 15.03 | 15.03 |
| Fat (g/100 g diet) | 4 | 9.8 | 9.8 |

MD = maltodextrin; IMOs = isomalto-oliogsaccharides; FOS = fructooligosaccharides; Inulin:FOS = 1:1 mixture of inulin and fructooligosaccharides.

During the study, body weight, food intake, body composition, respiratory quotient, and key molecular markers of metabolism were analyzed.

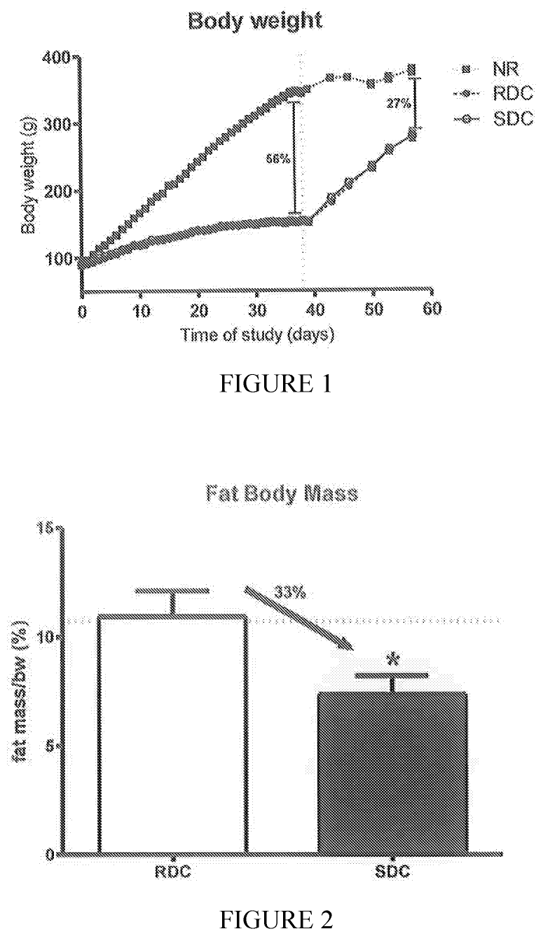

At the end of the food restriction period the data showed that the animals that received 70% of the diet consumed by the NR group had a large reduction in body weight gain as compared to the NR group (˜56% reduction), as seen in FIG. 1. During the refeeding period, dietary intervention induced a large increase in body weight gain (+80% increase). However, no differences were observed among the RDC group and the SDC group. Feed efficiency was similar among the groups.

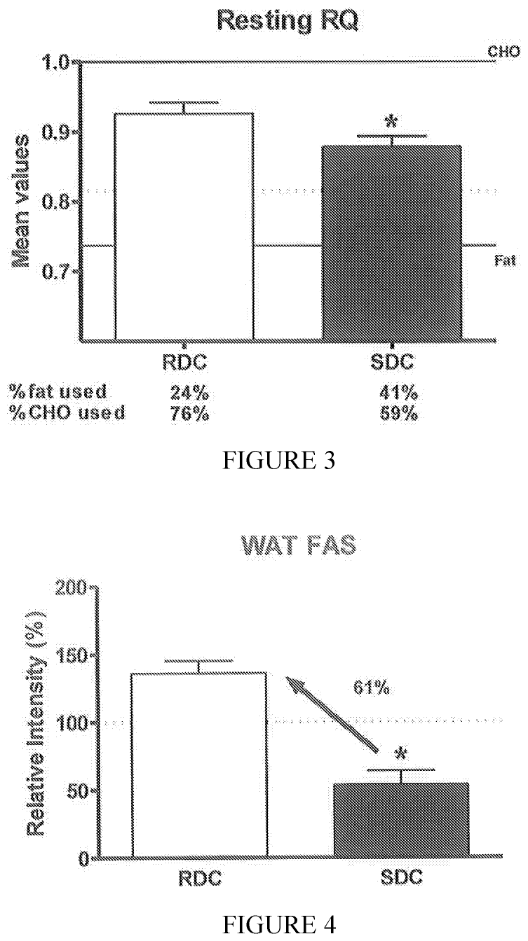

Regarding body composition, measurements by MRI at the end of the refeeding period revealed that the SDC group accumulated significantly less (˜33% less; p<0.05) fat than the RDC group, as seen in FIG. 2. The increase of fat depots in the RDC group could lead to the development of the catch-up fat phenotype.

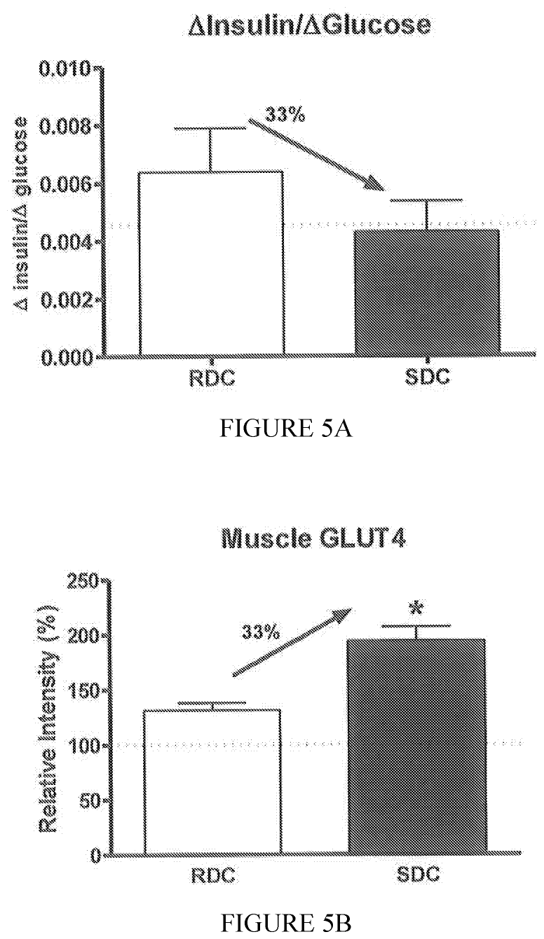

An important factor contributing to catch-up fat is reduced fat oxidation and increased carbohydrate oxidation. In order to determine the energy substrate utilization, respiratory quotient (RQ) was measured, and the results are illustrated in FIG. 3. The RQ is the ratio between the levels of oxygen consumption and carbon dioxide production, and reflects the ratio of carbohydrate oxidation to fat oxidation. A ratio close to 1:1 indicates increased carbohydrate oxidation over fat oxidation, whereas a lower ratio (close to 0.7:1) reflects an increased fatty acid oxidation relative to carbohydrate oxidation.

As seen in FIG. 3, the SDC group had significantly lower RQ values (p<0.05) as compared to the RDC group. Based on a modified Lusk table, the approximate use of each energy source was calculated for the RDC group and the SDC group. The RDC group primarily used carbohydrates (˜75%) as an energy source, whereas the SDC group maintained a better balance in the use of carbohydrates and fat as energy sources (˜59% carbohydrates and ˜41% fat).

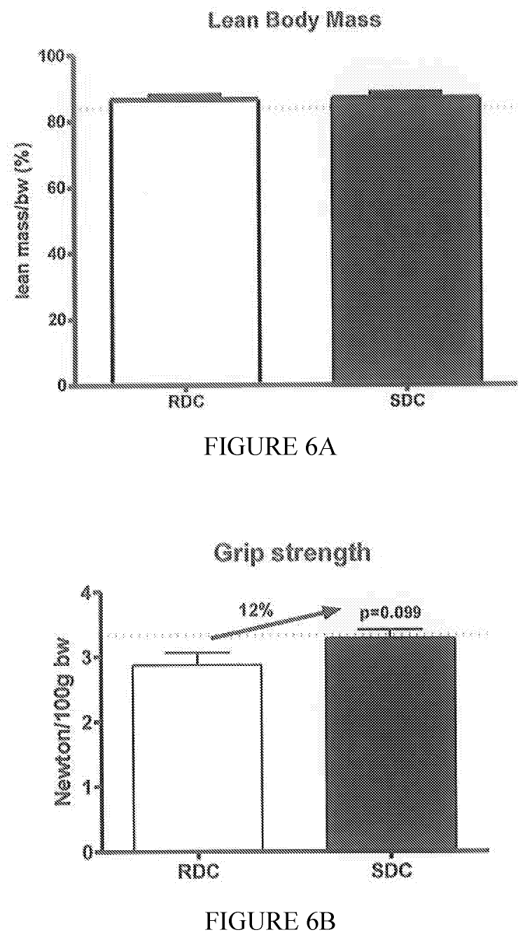

Because lipogenesis occurs in a RQ above 1.0, it is likely that the low RQ value of the SDC group reflects both relatively high fatty acid oxidation and low lipogenesis. This result was confirmed when the adipose tissue fatty acid synthase (FAS), a key enzyme involved in de novo lipogenesis, was analyzed. As shown in FIG. 4, the amount of FAS in the white adipose tissue (WAT) of the RDC group was significantly higher (˜61% higher; p<0.05) than that of the SDC group.

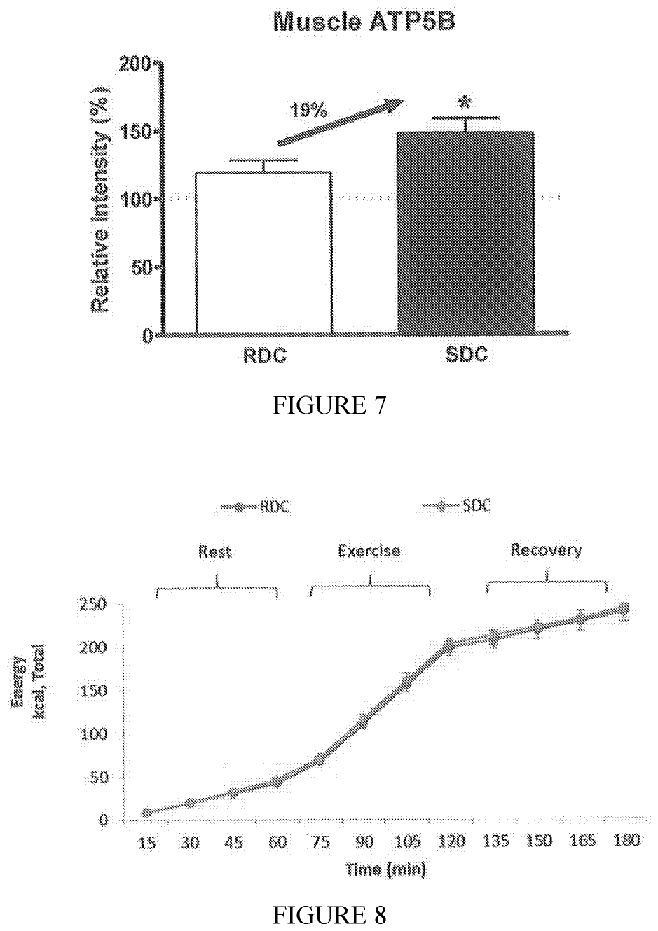

Previous studies have demonstrated that people who had a low birth weight or who were stunted during infancy presented an insulin-resistant state associated with a preferential acceleration of fat recovery or catch-up fat. In this study, at the end of the refeeding period, blood samples were isolated in fasting and in post-absorptive conditions (1 hour after an oral meal tolerance challenge with RDC and SDC diets; 10 kcal/kg bw). When Δblood insulin/Δblood glucose (Δ: 1-hour post-gavage value−fasting value) was calculated, the SDC group needed lower levels of circulating insulin than the RDC group to maintain normoglycemia, as seen in FIG. 5A. Moreover, molecular data obtained from muscle samples showed a significant increase (˜33% increase; p<0.05) in the expression of glucose transporter 4 (GLUT4) in the SDC group as compared to the RDC group, as seen in FIG. 5B, indicating a higher muscle glucose uptake. Both results lead to the conclusion that the consumption of a diet containing a carbohydrate blend of sources of rapidly available glucose, slowly available glucose, and non-digestible carbohydrates or resistant starch promotes higher sensitivity to insulin as compared to that induced by consumption of a diet containing a carbohydrate blend of sources of rapidly available glucose.

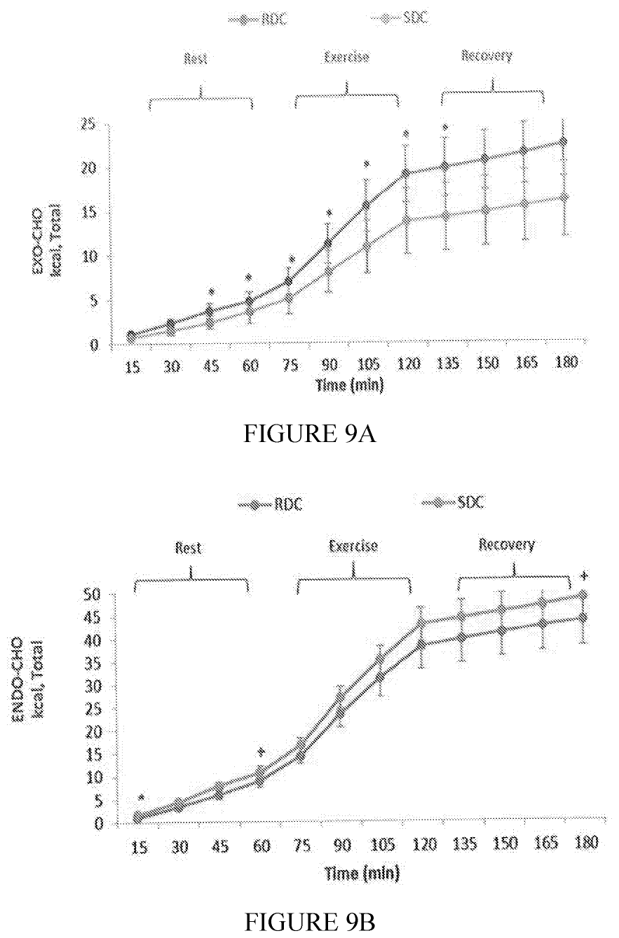

The forelimb grip strength of the RDC group and the SDC group was evaluated as an indicator of muscle function. Although soft lean body mass did not reveal any differences among the SDC group and the RDC group, as seen in FIG. 6A, the SDC group showed a trend toward higher grip strength values as compared to the RDC group (˜12% higher), as seen in FIG. 6B. These results indicate a positive effect on skeletal muscle function through the ingestion of the SDC diet during the refeeding period. Indeed, it is well known that a functional and active muscle increases GLUT4 expression to support an adequate glucose supply. Thus, the higher GLUT4 level promoted by the SDC diet (FIG. 5B) may contribute to improved muscle function (FIG. 6B).

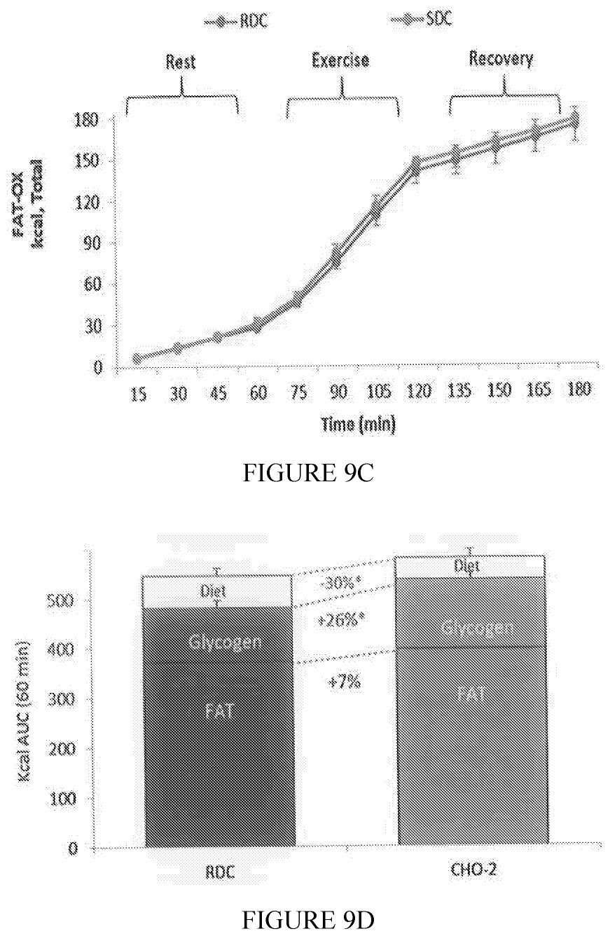

Another important factor for muscle functionality is an adequate adenosine triphosphate (ATP) supply. Mitochondria are responsible for the production of energy in the form of ATP by oxidative phosphorylation. A higher mitochondrial activity protects against ectopic fat accumulation and insulin resistance. Thus, ATP synthase (ATP5B) expression levels were assessed in muscle for the RDC group and the SDC group after the refeeding period. As seen in FIG. 7, the ATP5B levels of the SDC group were significantly higher (˜19% higher; p<0.05) than the ATP5B levels of the RDC group.

The study results indicate that diets containing a carbohydrate blend of sources of rapidly available glucose, slowly available glucose, and non-digestible carbohydrates or resistant starch, as present in the SDC diet, can reduce the development of catch-up fat and maintain adequate muscle performance. The healthy catch-up growth observed in the SDC group may be a direct result of improved metabolic flexibility, the ability to easily switch between carbohydrate oxidation and fat oxidation in response to homeostatic signals.

Example 2—A study was conducted to examine the effects of two carbohydrate blends (RDC and SDC) on postprandial metabolism and fat oxidation in pre-adolescent children. The study was a single-center, randomized, double-blind, cross-over, two treatment pilot study in a total of 19 subjects (n=19; 10 boys and 9 girls).

Over two study visits lasting 180 minutes each, subjects consumed (in random order) a single 8 fluid ounce serving of a lemonade-flavored study product containing 30 grams of a RDC carbohydrate blend and an SDC carbohydrate blend. The RDC carbohydrate blend contains sources of carbohydrate that provide rapidly available glucose. The SDC carbohydrate blend contains a source of carbohydrate that provides rapidly available glucose, a source of carbohydrate that provides slowly available glucose, and a source of non-digestible carbohydrate or resistant starch in accordance with the present invention. The compositions of the RDC and SDC carbohydrate blends are provided in Table 3.

After product consumption, subjects were studied during 60 minutes of rest before and after 60 minutes of physical activity on a cycle ergometer (standardized to 50% VO2 max). The total testing period was 180 minutes.

During each test, indirect calorimetry was used to assess oxygen (02) consumption and carbon dioxide (CO₂) production. Inspiratory and ventilatory gases were collected every 15 minutes for 180 minutes using a metabolic cart, facemask, pneumotachometer, and 02 and CO₂ analyzer. Samples of gases were collected for analysis of ¹²C and ¹³C for assessment of endogenous (glycogen) versus exogenous (diet) carbohydrate oxidation by gas chromatography/mass spectrometry. Fat and carbohydrate oxidation and energy expenditure were calculated using the following standard stoichiometric equations:

Carbohydrate Oxidation (g/min)=4.55*VCO₂−3.21*VO₂−2.87*n

Fat Oxidation (g/min)=1.67*VO₂−1.67*VCO₂−1.92*n

where VO₂ is the rate of oxygen consumption, VCO₂ is the rate of carbon dioxide production, and n is the rate of nitrogen excretion (assumed to be negligible).

TABLE 3
Composition of carbohydrate blends

| Carbohydrate | RDC | SDC | |
|---|---|---|---|
| Sucrose | 50 wt % | | |
| Isomaltulose | | 26.4 | wt % |
| Sucromalt | | 22.1 | wt % |
| Maltodextrin | 50 wt % | 23 | wt % |
| Isomalto-oligosaccharides | | 11.5 | wt % |
| Resistant Starch | | 10 | wt % |
| Inulin:FOS | | 7 | wt % |

Inulin:FOS = 1:1 mixture of inulin and fructooligosaccharides.

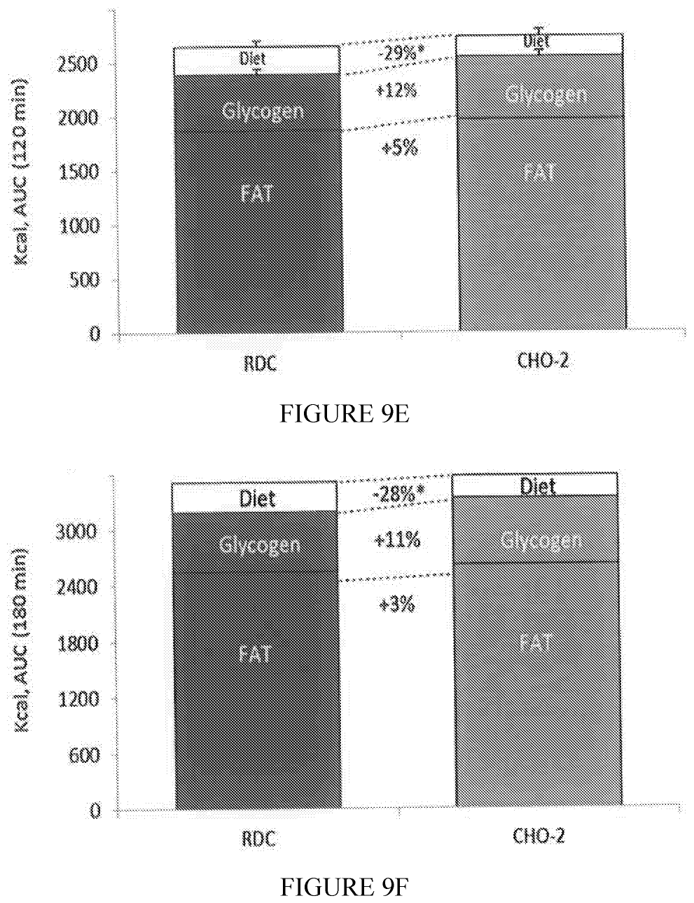

As seen in FIG. 8, there was no difference in total energy expenditure between treatments with the RDC carbohydrate blend and the SDC carbohydrate blend across the 180-minute postprandial study period. However, the differences in substrate utilization show significantly more stored energy (glycogen+fat) and less diet energy from carbohydrates were utilized to meet energy needs after consuming the SDC carbohydrate blend as compared to the RDC carbohydrate blend, as seen in Table 4.

TABLE 4
Ratio of Stored:Diet energy utilized across each study phase

| | Stored¹:Diet² Ratio | | |
|---|---|---|---|
| Study Phase | RDC | SDC | p value |
| Rest | 29 ± 7 | 48 ± 12 | p < 0.05 |
| Exercise | 40 ± 15 | 65 ± 23 | p < 0.05 |
| Recovery | 28 ± 6 | 45 ± 10 | p < 0.05 |

¹Stored energy is the AUC of energy from both glycogen and fat.
²Diet energy is the AUC of energy from the RDC or the SDC carbohydrate blend.

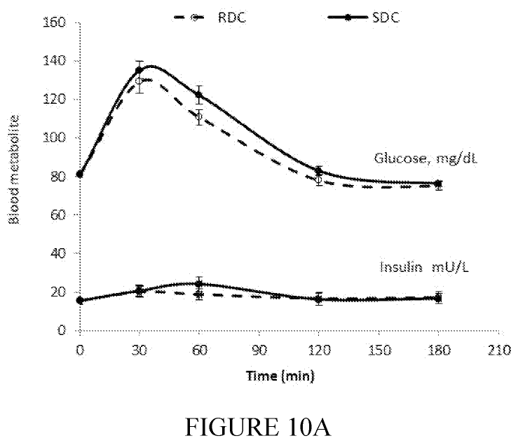

As shown in FIG. 9A, less exogenous energy (i.e., energy from consuming carbohydrate blend) was utilized after consuming the SDC carbohydrate blend as compared to the RDC carbohydrate blend (p<0.05 at most time points). FIG. 9B shows that more endogenous carbohydrate energy (stored glycogen) was utilized after consuming the SDC carbohydrate blend as compared to the RDC carbohydrate blend. Similarly, FIG. 9C shows that slightly more endogenous fat energy was utilized after consuming the SDC carbohydrate blend as compared to the RDC carbohydrate blend.

As seen in FIGS. 9D-9F, consumption of the SDC carbohydrate blend (labelled “CHO-2”) resulted in a 28-30% lower utilization of exogenous energy as compared to consumption of the RDC carbohydrate blend. The utilization of endogenous energy from carbohydrates (stored glycogen) was significantly higher (+26% increase; p<0.05) during the initial postprandial phase after consuming the SDC carbohydrate blend (CHO-2), as shown in FIG. 9D, but was not significantly higher during exercise (FIG. 9E) or after exercise (FIG. 9F), possibly due to both the marked increase in energy needs combined with a limited sample size. Fat oxidation was generally higher after consumption of the SDC carbohydrate blend (labelled “CHO-2”) as compared to consumption of the RDC carbohydrate blend, but the differences were not statistically significant, possibly due to the small sample size.

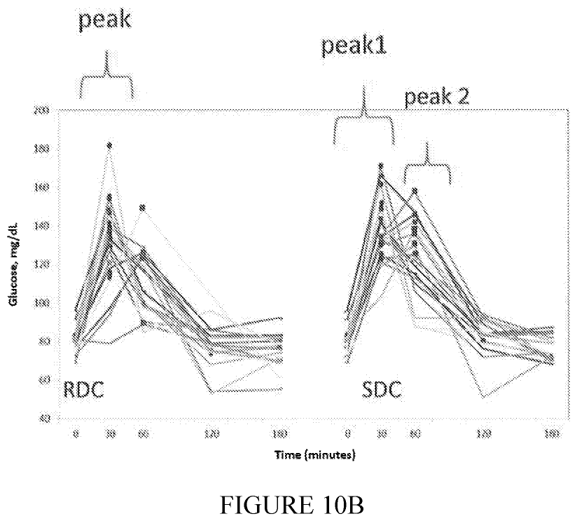

Of interest and unexpectedly, significant differences were observed in capillary glucose and insulin when assessed using finger-stick blood samples, as seen in FIG. 10A. As seen in FIG. 10B, individual glucose responses illustrate two glucose peaks 30 minutes and 60 minutes after consumption of the SDC carbohydrate blend, which suggests slower digestion/metabolism kinetics.

The study data demonstrate that, within a short period of time after feeding, the SDC carbohydrate blend can shift energy utilization toward the use of more stored or endogenous energy. Of significance is that these changes were observed after only one 30 gram serving of a carbohydrate blend, which is equivalent to the amount of carbohydrates present in a single serving of an oral nutritional supplement.

Unless otherwise indicated herein, all sub-embodiments and optional embodiments are respective sub-embodiments and optional embodiments to all embodiments described herein. While the present disclosure has been illustrated by the description of embodiments thereof, and while the embodiments have been described in considerable detail, it is not the intention of the applicant to restrict or in any way limit the scope of the appended claims to such detail. Additional advantages and modifications will readily appear to those skilled in the art. Therefore, the present disclosure, in its broader aspects, is not limited to the specific details, the representative compositions or formulations, and illustrative examples shown and described. Accordingly, departures may be made from such details without departing from the spirit or scope of the applicant's general disclosure herein.

1. A method of treating malnourishment in a moderately malnourished individual, the method comprising:
administering to the moderately malnourished individual a nutritional composition comprising a carbohydrate blend of:
(i) a source of at least one carbohydrate that provides rapidly available glucose;
(ii) a source of at least one carbohydrate that provides slowly available glucose; and
(iii) a source of at least one non-digestible carbohydrate or resistant starch.

2-4. (canceled)

5. The method according to claim 1, wherein the source of at least one carbohydrate that provides rapidly available glucose comprises one or more of:
(i) a monosaccharide;
(ii) a glucose unit and a fructose unit joined by an α-1, β-2 glycosidic linkage;
(iii) a glucose unit and a galactose unit joined by a β (1,4) glycosidic linkage;
(iv) glucose units joined by α (1,4) glycosidic linkages;
(v) glucose units joined by α (1,6) glycosidic linkages; and
(vi) oligosaccharides having a random mixture of α (1,2), α (1,3), α (1,4), and α (1,6) glycosidic linkages.

6. The method according to claim 5, wherein the source of at least one carbohydrate that provides rapidly available glucose comprises one or more of glucose, fructose, tagatose, galactose, mannose, ribose, sucrose, maltose, isomaltose, lactose, isomalto-oligosaccharides, maltodextrin, and starch.

7. The method according to claim 1, wherein the source of at least one carbohydrate that provides slowly available glucose comprises one or more of:
(i) a glucose unit and a fructose unit joined by an α (1,6) glycosidic linkage;
(ii) two glucose units joined by an α (1,1) glycosidic linkage;
(iii) a glucose unit and a fructose unit joined by an α (1,5) glycosidic linkage; and
(iv) oligosaccharides having alternating α (1,3) and α (1,6) glycosidic linkages.

8. The method according to claim 7, wherein the source of at least one carbohydrate that provides slowly available glucose comprises one or more of isomaltulose, trehalose, sucromalt, and leucrose.

9. The method according to claim 1, wherein the source of at least one non-digestible carbohydrate or resistant starch comprises one or more of:
(i) oligosaccharides having a random mixture of α (1,2), α (1,3), α (1,4), and β glycosidic linkages;
(ii) saccharides having linear chains of 2 to 60 fructose units or fructose polymers joined by β (2,1) glycosidic linkages; and
(iii) oligosaccharides having a random mixture of α (1,2), α (1,3), α (1,4), and α (1,6) glycosidic linkages.

10. The method according to claim 9, wherein the source of at least one non-digestible carbohydrate or resistant starch comprises one or more of resistant maltodextrin, fructooligosaccharides, inulin, and isomalto-oligosaccharides.

11. The method according to claim 1, wherein the source of at least one carbohydrate that provides rapidly available glucose provides from 4% to 71% of the total calories supplied by carbohydrates in the nutritional composition, the source of at least one carbohydrate that provides slowly available glucose provides from 25% to 86% of the total calories supplied by carbohydrates in the nutritional composition, and the source of at least one non-digestible carbohydrate or resistant starch provides from 3% to 20% of the total calories supplied by carbohydrates in the nutritional composition.

12. (canceled)

13. The method according to claim 1, wherein the moderately malnourished individual suffers from one or more of stunting and wasting.

14. The method according to claim 13, wherein the moderately malnourished individual is an infant or a preterm infant who suffers from stunting, and the stunting is caused by at least one of malnutrition, undernutrition, poor gut health, infectious disease, and compromised immune function.

15. A method of promoting healthy catch-up growth in a moderately malnourished individual, the method comprising:
administering to the moderately malnourished individual a nutritional composition comprising a carbohydrate blend of:
(i) a source of at least one carbohydrate that provides rapidly available glucose;
(ii) a source of at least one carbohydrate that provides slowly available glucose; and
(iii) a source of at least one non-digestible carbohydrate or resistant starch.

16. The method of claim 15, wherein the source of at least one carbohydrate that provides rapidly available glucose comprises one or more of:
(i) a monosaccharide;
(ii) a glucose unit and a fructose unit joined by an α-1, β-2 glycosidic linkage;
(iii) a glucose unit and a galactose unit joined by a β (1,4) glycosidic linkage;
(iv) glucose units joined by α (1,4) glycosidic linkages;
(v) glucose units joined by α (1,6) glycosidic linkages; and
(vi) oligosaccharides having a random mixture of α (1,2), α (1,3), α (1,4), and α (1,6) glycosidic linkages.

17. The method of claim 16, wherein the source of at least one carbohydrate that provides rapidly available glucose comprises one or more of glucose, fructose, tagatose, galactose, mannose, ribose, sucrose, maltose, isomaltose, lactose, isomalto-oligosaccharides, maltodextrin, and starch.

18. The method of claim 15, wherein the source of at least one carbohydrate that provides slowly available glucose comprises one or more of:
(i) a glucose unit and a fructose unit joined by an α (1,6) glycosidic linkage;
(ii) two glucose units joined by an α (1,1) glycosidic linkage;
(iii) a glucose unit and a fructose unit joined by an α (1,5) glycosidic linkage; and
(iv) oligosaccharides having alternating α (1,3) and α (1,6) glycosidic linkages.

19. The method of claim 18, wherein the source of at least one carbohydrate that provides slowly available glucose comprises one or more of isomaltulose, trehalose, sucromalt, and leucrose.

20. The method of claim 15, wherein the source of at least one non-digestible carbohydrate or resistant starch comprises one or more of:
(i) oligosaccharides having a random mixture of α (1,2), α (1,3), α (1,4), and β glycosidic linkages;
(ii) saccharides having linear chains of 2 to 60 fructose units or fructose polymers joined by β (2,1) glycosidic linkages; and
(iii) oligosaccharides having a random mixture of α (1,2), α (1,3), α (1,4), and α (1,6) glycosidic linkages.

21. The method of claim 20, wherein the source of at least one non-digestible carbohydrate or resistant starch comprises one or more of resistant maltodextrin, fructooligosaccharides, inulin, and isomalto-oligosaccharides.

22. The method of claim 15, wherein the source of at least one carbohydrate that provides rapidly available glucose provides from 4% to 71% of the total calories supplied by carbohydrates in the nutritional composition, the source of at least one carbohydrate that provides slowly available glucose provides from 25% to 86% of the total calories supplied by carbohydrates in the nutritional composition, and the source of at least one non-digestible carbohydrate or resistant starch provides from 3% to 20% of the total calories supplied by carbohydrates in the nutritional composition.

23-24. (canceled)

25. The method of claim 15, wherein the moderately malnourished individual suffers from one or more of stunting and wasting.

26. The method of claim 25, wherein the moderately malnourished individual is an infant or a preterm infant who suffers from stunting, and the stunting is caused by at least one of malnutrition, undernutrition, poor gut health, infectious disease, and compromised immune function.

27-51. (canceled)
